(19) United States
(12) Patent Application Publication
(10) Pub. No.: US 2020/0330497 A1
(43) Pub. Date: Oct. 22, 2020

(54) PREVENTION AND TREATMENT OF TDP-43 ASSOCIATED DISEASES

(71) Applicant: Elysium Health, Inc., New York, NY (US)

(72) Inventors: Eric A. Marcotulli, New York, NY (US); Dan Alminana, New York, NY (US); Mark Morris, New York, NY (US); Ryan Dellinger, Azusa, CA (US)

(21) Appl. No.: 16/757,037

(22) PCT Filed: Oct. 19, 2018
(86) PCT No.: PCT/US2018/056658

§ 371 Date: Apr. 17, 2020

Related U.S. Application Data
(60) Provisional application No. 62/574,477, filed on Oct. 19, 2017.

(51) Int. Cl. A61K 31/706; G01N 33/50; A61K 9/00; A61K 31/09

(52) U.S. Cl. CPC A61K 31/706; A61K 31/09; A61K 9/0053; G01N 33/5091

(57) ABSTRACT

Provided herein are methods and compositions related to treating and/or preventing diseases or disorder associated with cytoplasmic accumulation of TDB-43 by administering to the subject (e.g., orally administering to the subject) a composition comprising nicotinamide riboside and/or pterostilbene.

### CROSS-REFERENCE TO RELATED APPLICATIONS

This application claims the benefit of U.S. Provisional Application No. 62/574,477, filed on Oct. 19, 2017; hereby incorporated by reference in its entirety.

### BACKGROUND

Many neurodegenerative diseases are characterized by the deposition of insoluble protein in cells of the neuromuscular system. Advances in molecular neuropathology have allowed a classification system of neurodegenerative diseases based on insoluble protein accumulation. Microtubule-associated tau is one protein that has important functions in healthy neurons, but forms insoluble deposits in diseases now known collectively as tauopathies. Tauopathies encompass more than 20 clinicopathological entities, including amyotrophic lateral sclerosis ALS, progressive supranuclear palsy, Pick's disease, corticobasal degeneration and post-encephalitic parkinsons. Recent studies show that almost all cases of ALS and many other tauopathies share a common neuropathology characterized by the deposition of TAR-DNA binding protein (TDP)-43-positive protein inclusions.

Despite recent advances, therapeutic options for patients with TDP-43-associated diseases and disorders are limited. Hence, there is a need to develop new and efficacious therapies for such diseases and disorders.

### SUMMARY

Provided herein are methods and compositions related to treating diseases or disorders associated with the cytoplasmic accumulation of TDP-43 by administering to a subject (e.g., orally administering to the subject) a composition comprising a compound of Formula I or Formula II (e.g., nicotinamide riboside), and/or a compound of Formula III (e.g., pterostilbene). In some embodiments, the subject exhibits cytoplasmic accumulation of TDP-43 (e.g., at least 1%, at least 5%, at least 10%, at least 20%, at least 30%, at least 40%, or at least 50% of total TDP-43 in at least some of the neurons in a subject is in the cytoplasm of the neurons) (e.g., in neurons in the subject). In some embodiments, the subject carries a mutation associated with cytoplasmic accumulation of TDP-43. In some embodiments, the subject carries a TDP-43 variant protein associated with increased cytoplasmic accumulation.

In some embodiments, the disease or disorder is Alzheimer's disease, amyotrophic lateral sclerosis, parkinsonism dementia complex of Guam, argyrophilic grain disease, dementia pugilistica, chronic traumatic encephalopathy, diffuse neurofibrillary tangles with calcification, Down's syndrome, familial British dementia, familial Danish dementia, frontal lobe dementia, frontotemporal lobular degeneration, Gertmann-Straussler-Scheinker disease, globular glial taupathies, white matter taupathy with globular glial inclusions, Guadeloupean parkinsonism with dementia, Guadeloupean progressive supranuclear palsy, multiple system atrophy, myotonic dystrophy, neurodegeneration with brain iron accumulation, Hallevorden-Spatz disease, pantothenate kinase-associated neurodegeneration, neurofibrillary tangle-predominant dementia, Niemann-Pick disease, type C, Picks's disease, prosencephalitic parkinsonism, prion protein cerebral amyloid angiopathy, progressive supranuclear palsy, SLC9 A6-related mental retardation, or subacute sclerosing panecephalitis.

In some embodiments, the methods disclosed herein comprise testing analyzing the amount of TDP-43 in the cytoplasm in cells in a sample obtained from a subject (e.g., a cerebrospinal sample). In certain embodiments, the methods provided herein comprise determining whether a sample obtained from a subject comprises nucleic acid (e.g., DNA or RNA) encoding a mutation associated with increased cellular accumulation of TDP-43. In some embodiments, the method comprises testing a sample obtained from a subject (e.g., a cerebrospinal sample) for the presence of a TDP-43 variant protein associated with increased cytoplasmic accumulation. In some embodiments, if increased cytoplasmic accumulation of TDP-43, the presence of a nucleic acid encoding a mutation associated with increased cytoplasmic accumulation of TDP-43 and/or a TDP-43 protein variant associated with increased cytoplasmic accumulation of TDP-43 is detected, the subject is selected for treatment with a composition comprising a compound of Formula I or Formula II (e.g., nicotinamide riboside), and/or a compound of Formula III (e.g., pterostilbene). In some embodiments, if the subject is selected for treatment with the composition, the subject is administered the composition.

In some embodiments, determining the amount of TDP-43 in the cytoplasm comprises contacting the cells with an antibody specific for TDP-43. In some embodiments, TDP-43 has accumulated in the cytoplasm if at least 1%, at least 5%, at least 10%, at least 20%, at least 30%, at least 40%, or at least 50% of total TDP-43 in the cells of the sample is in the cytoplasm of the cells. In other embodiments, TDP-43 has accumulated in the cytoplasm if the amount of TDP-43 in the cytoplasm in the cells of the sample is greater than a sample from a healthy subject.

Also provided herein are methods of selecting a subject for treatment with a composition comprising a compound of Formula I or Formula II (e.g., nicotinamide riboside), and/or a compound of Formula III (e.g., pterostilbene). In some embodiments, the selecting a subject for therapy comprises obtaining a sample (e.g., a cerebrospinal sample) comprising cells from the subject and identifying a genetic mutation in the DNA in the sample and, if the DNA has the genetic mutation, selecting the subject for therapy comprising administration of a composition comprising a compound of Formula I or Formula II (e.g., nicotinamide riboside), and/or a compound of Formula III (e.g., pterostilbene). In some embodiments, the genetic mutation is in the TARDBP. In certain embodiments, the genetic mutation is a truncation mutation. In certain embodiments, the mutation may be any genetic mutation that promotes the mislocalization of nuclear TDP-43 to the cytoplasm.

In some embodiments, the methods provided herein include determining whether a pathological form of TDP-43 is present in a sample from the subject. In some embodiments, the method includes administering to the subject a composition comprising a compound of Formula I or Formula II (e.g., nicotinamide riboside), and/or a compound of Formula III (e.g., pterostilbene). In some embodiments, the pathological form of TDP-43 is phosphorylated TDP-43 or a truncated form of TDP-43. A pathological form of TDP-43 may be any mutation that promotes mislocalization of nuclear TDP-43 to the cytoplasm or that otherwise promotes the accumulation of TDP-43 in the cytoplasm. Identifying a pathological form of TDP-43 may comprise contacting the cells in the sample with an antibody specific for the pathological form of TDP-43.

In certain embodiments, the composition comprises a compound of Formula I or Formula II (e.g., at least 400 mg, at least 500 mg, at least 600 mg, at least 700 mg, at least 800 mg, at least 900 mg, at least 1000 mg, at least 1100 mg, at least 1200 mg, at least 1300 mg, at least 1400 mg, at least 1500 mg, at least 1600 mg, at least 1700 mg, at least 1800 mg, at least 1900 mg, at least 2000 mg, at least 2100 mg, at least 2200 mg, at least 2300 mg or at least 2400 mg of a compound of Formula I or Formula II). In some embodiments, the composition comprises a compound of formula III (e.g., at least 60 mg, at least 80 mg, at least 100 mg, at least 120 mg, at least 140 mg, at least 160 mg, at least 180 mg, at least 200 mg, at least 220 mg, at least 240 mg, at least 260 mg, at least 280 mg, at least 300 mg, at least 320 mg, at least 340 mg, at least 360 mg, at least 380 mg, at least 400 mg, at least 500 mg, or at least 600 mg of a compound of formula III). In certain embodiments, the composition comprises both a compound of Formula I or Formula II (e.g., at least 400 mg, at least 500 mg, at least 600 mg, at least 700 mg, at least 800 mg, at least 900 mg, at least 1000 mg, at least 1100 mg, at least 1200 mg, at least 1300 mg, at least 1400 mg, at least 1500 mg, at least 1600 mg, at least 1700 mg, at least 1800 mg, at least 1900 mg, at least 2000 mg, at least 2100 mg, at least 2200 mg, at least 2300 mg, or at least 2400 mg of a compound of Formula I or Formula II) and a compound of Formula III (e.g., at least 60 mg, at least 80 mg, at least 100 mg, at least 120 mg, at least 140 mg, at least 160 mg, at least 180 mg, at least 200 mg, at least 220 mg, at least 240 mg, at least 260 mg, at least 280 mg, at least 300 mg, at least 320 mg, at least 340 mg, at least 360 mg, at least 380 mg, at least 400 mg, at least 500 mg, or at least 600 mg of a compound of formula III).

In certain embodiments, the method comprises administering a plurality of doses of the composition. In some embodiments, at least 7 doses of the composition are administered. In some embodiments, at least 30 doses of the composition are administered. In some embodiments, at least 60 or more doses of the composition are administered. In some embodiments, each dose comprises at least 400 mg, at least 500 mg, at least 600 mg, at least 700 mg, at least 800 mg, at least 900 mg, at least 1000 mg, at least 1100 mg, at least 1200 mg, at least 1300 mg, at least 1400 mg, at least 1500 mg, at least 1600 mg, at least 1700 mg, at least 1800 mg, at least 1900 mg, at least 2000 mg, at least 2100 mg, at least 2200 mg, at least 2300 mg, or at least 2400 mg of a compound of Formula I or Formula II (e.g., nicotinamide riboside). In some embodiments, each dose comprises at least 60 mg, at least 80 mg, at least 100 mg, at least 120 mg, at least 140 mg, at least 160 mg, at least 180 mg, at least 200 mg, at least 220 mg, at least 240 mg, at least 260 mg, at least 280 mg, at least 300 mg, at least 320 mg, at least 340 mg, at least 360 mg, at least 380 mg, at least 400 mg, at least 500 mg or at least 600 mg of a compound of formula III (e.g., pterostilbene).

In certain embodiments, each dose comprises at least 400 mg, at least 500 mg, at least 600 mg, at least 700 mg, at least 800 mg, at least 900 mg, at least 1000 mg, at least 1100 mg, at least 1200 mg, at least 1300 mg, at least 1400 mg, at least 1500 mg, at least 1600 mg, at least 1700 mg, at least 1800 mg, at least 1900 mg, at least 2000 mg, at least 2100 mg, at least 2200 mg, at least 2300 mg or at least 2400 mg of a compound of Formula I or Formula II (e.g., nicotinamide riboside) and at least 60 mg, at least 80 mg, at least 100 mg, at least 120 mg, at least 140 mg, at least 160 mg, at least 180 mg, at least 200 mg, at least 220 mg, at least 240 mg, at least 260 mg, at least 280 mg, at least 300 mg, at least 320 mg, at least 340 mg, at least 360 mg, at least 380 mg, at least 400 mg, at least 500 mg or at least 600 mg of a compound of Formula III (e.g., pterostilbene).

In certain embodiments, a dose of the composition is administered at regular intervals over a period of time. In some embodiments, a dose of the composition is administered at least once a week. In some embodiments, a dose of the composition is administered at least twice a week. In certain embodiments, a dose of the composition is administered at least three times a week. In some embodiments, a dose of the composition is administered at least once a day. In some embodiments, a dose of the composition is administered at least twice a day. In some embodiments, doses of the composition are administered for at least 1 week, for at least 2 weeks, for at least 3 weeks, for at least 4 weeks, for at least 1 month, for at least 2 months, for at least 3 months, for at least 4 months, for at least 5 months, for at least 6 months or for at least 1 year.

In certain embodiments, the composition is formulated for oral delivery. In some embodiments, the composition is formulated as a pill, a tablet, or a capsule. In some embodiments, the composition is administered orally. In certain embodiments, the composition is self-administered.

### DETAILED DESCRIPTION

### General

Provided herein are methods and compositions related to treating diseases or disorders associated with accumulation of TDP-43 in the cell cytoplasm by administering to a subject (e.g., orally administering to the subject) a composition comprising a compound of Formula I or Formula II (e.g., nicotinamide riboside), and/or a compound of Formula III (e.g., pterostilbene). Also provided herein are methods of selecting a subject for such therapy by detecting elevated cellular accumulation of TDP-43 in a subject, by detecting the presence of a mutation associated with elevated cellular accumulation of TDP-43 in a subject and/or by detecting the presence of a TDP-43 protein variant associated with elevated cellular accumulation of TDP-43 in a subject.

TAR DNA-binding protein-43 (TDP-43) is a nuclear protein functioning in the regulation of transcription and mRNA splicing. TDP-43 is accumulated in ubiquitinated inclusions in frontotemporal lobar degeneration with ubiquitin-positive inclusions (FTLD-U) and amyotrophic lateral sclerosis (ALS) diseased brains. Mislocalization of nuclear TDP-43 protein to the cytoplasm of the cell is characteristic of TDP-43 proteinopathies as well as many tauopathies.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

Unless otherwise specified here within, the terms “antibody” and “antibodies” broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a biomarker polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

As used herein, the term “administering” means providing a pharmaceutical agent or composition to a subject, and includes, but is not limited to, administering by a medical professional and self-administering. Administration of a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the term “monoclonal antibody” refers to an antibody obtained from a population of substantially homogeneous antibodies that specifically bind to the same epitope, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method.

The phrase “pharmaceutically-acceptable carrier” as used herein means a pharmaceutically-acceptable material, composition or vehicle, such as a liquid or solid filler, diluent, excipient, or solvent encapsulating material.

As used herein, the term “subject” means a human or non-human animal selected for treatment or therapy.

As used herein, a “TDP-43 proteinopathy” is a disease or disorder associated or characterized by the accumulation of TDP-43 in the cell cytoplasm or the mislocalization of nuclear TDP-43 to the cytoplasm of the cell. A TDP-43 proteinopathy may be characterized by aggregations of TDP-43 in a subject's CNS. In some embodiments, the TDP-43 proteinopathy is a tauopathy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

As used herein, a therapeutic that “prevents” a disorder or condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

Compositions

Provided herein are pharmaceutical compositions comprising a compound of Formula I or Formula II (e.g., nicotinamide riboside) and/or a compound of Formula III (e.g., pterostilbene).

Nicotinamide riboside is a pyridine-nucleoside form of niacin (i.e., vitamin B₃) that serves as a precursor to nicotinamide adenine dinucleotide (NAD⁺). As used herein, “nicotinamide riboside” also includes nicotinamide riboside salts, such as nicotinamide riboside chloride. The chemical structure of nicotinamide riboside is provided below:

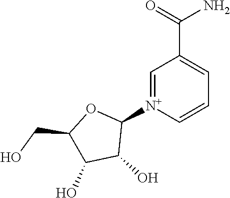 [SMILES: NC(=O)c1ccc[n+]([C@@H]2O[C@H](CO)[C@@H](O)[C@H]2O)c1]

In some embodiments, provided herein are pharmaceutical compositions comprising a compound represented by Formula (I) or a pharmaceutically acceptable salt thereof:

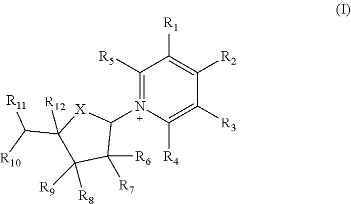

wherein, independently for each occurrence:

R₁, R₂, and R₃ are selected from hydrogen, halogen, —CN, —NO₂, —OR₁₄, —N(R₁₄)ₘ, —R₁₃, substituted or unsubstituted (C₁-C₆)alkyl, cycloalkyl, heterocycloalkyl, aryl, heteroaryl, aralkyl, and heteroaralkyl;

R₄ and R₅ are selected from hydrogen, halogen, —CN, —NO₂, —OR₁₄, —N(R₁₄)ₘ, substituted or unsubstituted (C₁-C₆)alkyl, cycloalkyl, heterocycloalkyl, aryl, heteroaryl, aralkyl, and heteroaralkyl;

R₆, R₈, R₁₁, and R₁₂ are selected from hydrogen, (C₁-C₆)alkyl, —((C₁-C₆)alkylene)N(R₁₄)ₘ, —C(O)((C₁-C₆)alkylene)N(R₁₄)ₘ, cycloalkyl, heterocycloalkyl, aryl, heteroaryl, aralkyl, heteroaralkyl, —OR₁₄, and —N(R₁₄)ₘ;

R₇, R₉, and R₁₀ are selected from —((C₁-C₆)alkylene)N(R₁₄)ₘ, —OR₁₄, and —N(R₁₄)ₘ;

R₁₃ is selected from —OR₁₄, —N(R₁₄)ₘ, —C(O)(R₁₄), —C(O)(OR₁₄), —C(O)N(R₁₄)ₘ, —₄), —S(O)OR₁₄, and —S(O)₂N(R₁₄)ₘ;

R₁₄ is selected from hydrogen, (C₁-C₆)alkyl, cycloalkyl, heterocycloalkyl, aryl, heteroaryl, aralkyl, and heteroaralkyl; and

X is O, S, or N(R₁₄);

m is 2 or 3;

provided that at least one of R₁, R₂, and R₃ is R₁₃.

In some embodiments, R₁ is R₁₃. In some embodiments, R₂ is R₁₃. In some embodiments, R₃ is R₁₃.

In some embodiments, R₁₃ is selected from —OR₁₄, —N(R₁₄)ₘ, —C(O)(R₁₄), —C(O)(OR₁₄), and —C(O)N(R₁₄)ₘ. In some embodiments, R₁₃ is selected from —C(O)(R₁₄), —C(O)(OR₁₄), and —C(O)N(R₁₄)ₘ. In some embodiments, R₁₃ is —C(O)N(R₁₄)ₘ.

In some embodiments, R₇, R₉, and R₁₀ are each independently —OR₁₄ or —N(R₁₄)ₘ. In some embodiments, R₇, R₉, and R₁₀ are —OR₁₄.

In some embodiments, the compound of formula (I) is represented by Formula (II) or a pharmaceutically acceptable salt thereof:

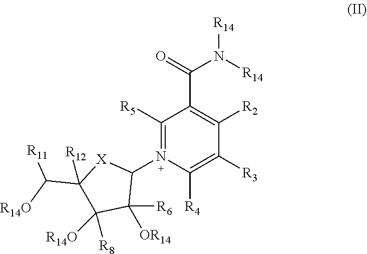

wherein, independently for each occurrence:

R₂ and R₃ are selected from hydrogen, halogen, —CN, —NO₂, —OR₁₄, —N(R₁₄)ₘ, —R₁₃, substituted or unsubstituted (C₁-C₆)alkyl, cycloalkyl, heterocycloalkyl, aryl, heteroaryl, aralkyl, and heteroaralkyl;

R₄ and R₅ are selected from hydrogen, halogen, —CN, —NO₂, —OR₁₄, —N(R₁₄)ₘ, substituted or unsubstituted (C₁-C₆)alkyl, cycloalkyl, heterocycloalkyl, aryl, heteroaryl, aralkyl, and heteroaralkyl;

R₆, R₈, R₁₁, and R₁₂ are selected from hydrogen, —OR₁₄, —N(R₁₄)ₘ, substituted or unsubstituted (C₁-C₆)alkyl, —((C₁-C₆)alkylene)N(R₁₄)ₘ, —C(O)((C₁-C₆)alkylene)N(R₁₄)ₘ, cycloalkyl, heterocycloalkyl, aryl, heteroaryl, aralkyl, and heteroaralkyl;

R₁₃ is selected from —OR₁₄, —N(R₁₄)ₘ, —C(O)(R₁₄), —C(O)(OR₁₄), —C(O)N(R₁₄)ₘ, —S(O)₂(OR₁₄), —S(O)OR₁₄, and —S(O)₂N(R₁₄)ₘ;

R₁₄ is selected from hydrogen, (C₁-C₆)alkyl, cycloalkyl, heterocycloalkyl, aryl, heteroaryl, aralkyl, and heteroaralkyl; and

m is 2 or 3.

In some embodiments of the compounds of formula (I) or (II), R₁, R₂, and R₃ are each independently, if present, selected from hydrogen, halogen, —CN, —NO₂, —OR₁₄, —N(R₁₄)ₘ, —R₁₃, and substituted or unsubstituted (C₁-C₆)alkyl. In some embodiments, R₁, R₂, and R₃ are each independently, if present, selected from hydrogen, —OR₁₄, —N(R₁₄)ₘ, and unsubstituted (C₁-C₆)alkyl. In some embodiments, R₁, R₂, and R₃ are each independently, if present, selected from substituted or unsubstituted (C₁-C₆)alkyl, cycloalkyl, heterocycloalkyl, aryl, heteroaryl, aralkyl, and heteroaralkyl. In some embodiments, R₁, R₂, and R₃ are each independently, if present, hydrogen.

In some embodiments of the compounds of formula (I) or (II), R₄ and R₅ are each independently selected from hydrogen, halogen, —CN, —NO₂, —OR₁₄, —N(R₁₄)ₘ, and substituted or unsubstituted (C₁-C₆)alkyl. In some embodiments, R₄ and R₅ are each independently selected from hydrogen, —OR₁₄, —N(R₁₄)ₘ, and unsubstituted (C₁-C₆)alkyl. In some embodiments, R₄ and R₅ are each independently selected from substituted or unsubstituted (C₁-C₆)alkyl, cycloalkyl, heterocycloalkyl, aryl, heteroaryl, aralkyl, and heteroaralkyl. In some embodiments, R₄ and R₅ are each hydrogen.

In some embodiments of the compounds of formula (I) or (II), R₆, R₈, R₁₁, and R₁₂ are selected from hydrogen, —OR₁₄, —N(R₁₄)ₘ, unsubstituted (C₁-C₆)alkyl, —((C₁-C₆)alkylene)N(R₁₄)ₘ, —C(O)((C₁-C₆)alkylene)N(R₁₄)ₘ, cycloalkyl, heterocycloalkyl, aryl, heteroaryl, aralkyl, and heteroaralkyl. In some embodiments, R₆, R₈, R₁₁, and R₁₂ are each independently selected from hydrogen, —OR₁₄, —N(R₁₄)ₘ, unsubstituted (C₁-C₆)alkyl, —((C₁-C₆)alkylene)N(R₁₄)ₘ, and —C(O)((C₁-C₆)alkylene)N(R₁₄)ₘ. In some embodiments, R₆, R₈, R₁₁, and R₁₂ are each independently selected from hydrogen, —OR₁₄, and —N(R₁₄)ₘ. In some embodiments, R₆, R₈, R₁₁, and R₁₂ are each independently selected from unsubstituted (C₁-C₆)alkyl, cycloalkyl, heterocycloalkyl, aryl, heteroaryl, aralkyl, and heteroaralkyl. In some embodiments, R₆, R₈, R₁₁, and R₁₂ are each hydrogen.

In some embodiments, R₇, R₉, and R₁₀ are each independently —OR₁₄ or —N(R₁₄)ₘ. In some embodiments, R₇, R₉, and R₁₀ are each —OR₁₄. In some embodiments, R₇, R₉, and R₁₀ are each —OH.

In some embodiments of the compounds of formula (I) or (II), R₁₄ is hydrogen or (C₁-C₆)alkyl.

In some embodiments of the compounds of formula (I) or (II), X is O or N(R₁₄). In some embodiments, X is O.

In some embodiments of the compounds of formula (I) or (II), the compound is

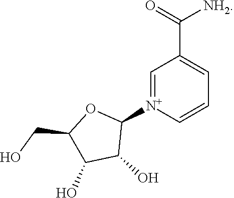 [SMILES: NC(=O)c1ccc[n+]([C@@H]2O[C@H](CO)[C@@H](O)[C@H]2O)c1]

Pterostilbene is a stilbenoid and an analog of polyphenol reservatrol that has better bioavailability due to the presence of two methoxy groups that allow it to have increased lipophilic and oral absorption as well as a longer half-life due to reduced oxidation. The chemical structure of pterostilbene is provided below:

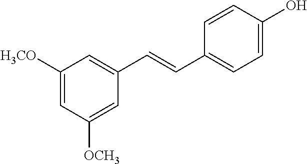 [SMILES: COc1cc(/C=C/c2ccc(O)cc2)cc(OC)c1]

In some embodiments, provided herein are pharmaceutical compositions comprising a compound represented by Formula (III) or a pharmaceutically acceptable salt thereof:

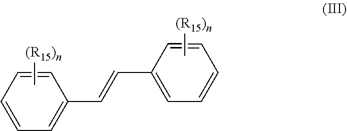

wherein, independently for each occurrence:

R₁₅ is selected from halogen, —CN, —NO₂, —OR₁₆, —N(R₁₆)ₚ, —S(O)₂(OR₁₆), —S(O)OR₁₆, substituted or unsubstituted (C₁-C₆)alkyl, cycloalkyl, heterocycloalkyl, aryl, heteroaryl, aralkyl, and heteroaralkyl;

R₁₆ is selected from hydrogen, (C₁-C₆)alkyl, cycloalkyl, heterocycloalkyl, aryl, heteroaryl, aralkyl, and heteroaralkyl;

n is an integer from 0 to 5; and

p is 2 or 3;

provided that at least one n is 1; and at least one R₁₅ is —OR₁₆;

provided that the compound of formula (III) is not

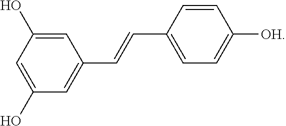 [SMILES: Oc1ccc(/C=C/c2cc(O)cc(O)c2)cc1]

In some embodiments of the compounds of formula (III), R₁₅ is selected from, halogen, —CN, —NO₂, —OR₁₆, —N(R₁₆)ₚ, and substituted or unsubstituted (C₁-C₆)alkyl. In some embodiments, R₁₅ is selected from —OR₁₆, —N(R₁₆)ₚ, and unsubstituted (C₁-C₆)alkyl. In some embodiments, R₁₅ is selected from substituted or unsubstituted (C₁-C₆)alkyl, cycloalkyl, heterocycloalkyl, aryl, heteroaryl, aralkyl, and heteroaralkyl. In some embodiments, R₁₅ is —OR₁₆. In some embodiments, R₁₅ is —OR₁₆; and R₁₆ is hydrogen or (C₁-C₆)alkyl. In some embodiments, R₁₃ is —OR₁₆; and R₁₆ is (C₁-C₆)alkyl, cycloalkyl, heterocycloalkyl, aryl, heteroaryl, aralkyl, and heteroaralkyl. In some embodiments, R₁₅ is —OR₁₆; and R₁₆ is (C₁-C₆)alkyl. In some embodiments, R₁₅ is —OR₁₆; and R₁₆ is (C₁-C₆)alkyl, cycloalkyl, or heterocycloalkyl.

In some embodiments, n is 1, 2, or 3. In some embodiments, n is 1 or 2.

In some embodiments, p is 2. In some embodiments, p is 3.

In one aspect, the provided herein are pharmaceutically acceptable compositions which comprise a therapeutically-effective amount of one or more of the compounds described herein (e.g., nicotinamide riboside and/or pterostilbene), formulated together with one or more pharmaceutically acceptable carriers (additives) and/or diluents. In another aspect, the agents described herein can be administered as such, or administered in mixtures with pharmaceutically acceptable carriers and can also be administered in conjunction with other agents. Conjunctive therapy thus includes sequential, simultaneous and separate, or co-administration of one or more compounds of the invention, wherein the therapeutic effects of the first administered has not entirely disappeared when the subsequent compound is administered.

As described in detail below, the pharmaceutical compositions described herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; or (3) sublingually.

In some embodiments, the composition comprises additional agents. For example, the composition may comprise a nutritional agent, such as an antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The formulations of the compounds described herein may be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the agent which produces a therapeutic effect.

In certain embodiments, a formulation described herein comprises an excipient, including, but not limited to, cyclodextrins, liposomes, micelle forming agents, e.g., bile acids, and polymeric carriers, e.g., polyesters and polyanhydrides; and an agent of the invention. In some embodiments, an aforementioned formulation renders orally bioavailable an agent of the invention. Methods of preparing these formulations or compositions may include the step of bringing into association a compound of the invention with the carrier and, optionally, one or more accessory ingredients.

Liquid dosage forms for oral administration of the formulations provided herein include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations provided herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention as an active ingredient. A compound of the invention may also be administered as a bolus, electuary, or paste.

In solid dosage forms of the invention for oral administration (e.g., capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions described herein, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. Compositions described herein may also be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Pharmaceutical compositions provided herein suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Therapeutic Methods

Provided herein are methods and compositions related to treating and/or preventing diseases or disorders associated with TDP-43 proteinopathies and/or tauopathies in a subject by obtaining a sample (e.g., a sample with biological tissue, such as a cerebrospinal sample) comprising cells, analyzing the relative level of TDP-43 in the cytoplasm of the cells in the sample and, if the subject has accumulation of TDP-43 in the cytoplasm, administering to the subject (e.g., orally administering to the subject) a composition comprising a compound of Formula I or Formula II (e.g., nicotinamide riboside), and/or a compound of Formula III (e.g., pterostilbene). Also provided herein are methods of selecting a subject for therapy comprising administration of a composition comprising a compound of Formula I or Formula II (e.g., nicotinamide riboside), and/or a compound of Formula III (e.g., pterostilbene) the method comprising obtaining a sample comprising cells from the subject, analyzing the cytoplasmic levels of TDP-43 in the cells in the sample and, if TDP-43 has accumulated in the cytoplasm of the cells, and selecting the subject for therapy comprising administration of a composition comprising a compound of Formula I or Formula II (e.g., nicotinamide riboside), and/or a compound of Formula III (e.g., pterostilbene).

A TDP-43 proteinopathy may refer to any disease or disorder that is associated with the mislocalization of nuclear TDP-43 protein to the cytoplasm and/or accumulation of TDP-43 protein in the cytoplasm. In some embodiments, the TDP-43 proteinopathy is a tauopathy. Examples of tauopathies include, but are not limited to, Alzheimer's disease (AD), myotrophic lateral sclerosis, parkinsonism-dementia complex of Guam, argyrophilic grain disease, chronic traumatic encephalopathy/dementia pugilistica, corticobasal degeneration, diffuse neurofibrillary tangles with calcification, Down's syndrome, familial British dementia, familial Danish dementia, frontal lobe dementia (non-AD, non-pick), frontotemporal dementia and parkinsonism linked to chromosome 17 (caused by MAPT mutations), frontotemporal lobar degeneration (non-AD, non-pick), frontal lobe dementia and parkinsonism linked to chromosome 17 caused by MAPT mutations, frontotemporal lobar degeneration caused by C9ORF72 mutations, Gerstmann-Sträussler-Scheinker disease, Globular glial tauopathies/white matter tauopathy with globular glial inclusions, Guadeloupean parkinsonism with dementia, Guadeloupean PSP, multiple system atrophy, myotonic dystrophy, neurodegeneration with brain iron accumulation, Niemann-Pick disease, type C, Pick's disease, postencephalitic parkinsonism, prion protein cerebral amyloid angiopathy, progressive supranuclear palsy, SLC9A6-related mental retardation, and subacute sclerosing panencephalitis.

The subject may be male or female. In some embodiments, the subject is an adult (i.e., 18 years of age or older). The subject may be pediatric (i.e., less than 18 years of age). In some embodiments, the subject is a mammal, preferably, a human. The subject may have a TDP-43 proteinopathy and/or a tauopathy, or may be at risk for a TDP-43 proteinopathy and/or a tauopathy. For example, the subject may have a familial history of a TDP-43 proteinopathy and/or a tauopathy.

A sample disclosed herein may be cerebrospinal tissue, i.e., tissue located in or derived from the brain or spinal cord or a combination thereof. If located in or derived from the spinal cord, the tissue may include or consist exclusively of cerebrospinal fluid (CSF). Thus, as used herein, “tissue” may be a biological fluid, such as blood, urine, saliva, cerebrospinal fluid, and the like. Within the brain, the tissue can be of the frontal cortex, temporal cortex, hippocampus, or brain stem, or a combination thereof. As used herein the phrase “a tissue of” refers both to tissue that is located in situ and to tissue that has been partially or fully moved within or extracted from the subject; as such, all manners of access to tissue are contemplated as being within the scope of the disclosed methods.

Also provided are methods of determining the amount of cytoplasmic TDP-43 in cell within a sample by contacting the sample with an antibody that binds to TDP-43 or a fragment thereof; and, determining the extent of binding of the antibody to the tissue and/or cells within the sample. Preferably, the sample and/or tissue with which the anti-TDP-43 antibody is contacted is tissue of the central nervous system (e.g., cerebrospinal tissue samples). Exemplary cerebrospinal tissue includes tissue of the hippocampus, neocortex, brain stem, and spinal cord.

In some embodiments, analyzing the cytoplasmic levels of TDP-43 comprise contacting sample with an antibody specific for TDP-43. The antibody may have a detectable label (i.e., a fluorescent label). Antibody binding may be determined, for example, through microscopy or any other method known in the art. In some embodiments, the protein from the cells within the sample may be isolated and contacted with a TDP-43 antibody. The TDP-43 antibody may be immobilized on a solid support. In some embodiments, the antibody is specific for wild-type TDP-43. In other embodiments, the TDP-43 antibody specifically binds a pathogenic form of TDP-43, such as a mutated or truncated form (e.g., TDP-25) of TDP-43. In some embodiments, the antibody is polyclonal. In some embodiments, the antibody is monoclonal. In some embodiments, the TDP-43 antibody can be of any species. In some embodiments, the antibody is a mouse, rat, sheep, goat, camel, chicken, duck, hamster, guinea pig, dog, monkey, human or synthetic antibody or a combination thereof.

The methods disclosed herein comprise analyzing or determining whether TDP-43 has accumulated in the cell cytoplasm. In some embodiments, TDP-43 has accumulated in the cytoplasm if at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, or at least 70% of total TDP-43 in the cells in the sample is in the cytoplasm of the cells.

In some embodiments, TDP-43 has accumulated in the cytoplasm if at least at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, or at least 70% of the cells in the sample have TDP-43 localized to the cytoplasm of the cell.

In some embodiments, TDP-43 has accumulated in the cytoplasm of the cells within the sample if the amount of TDP-43 in the cytoplasm of the cells within the sample is greater than the amount of TDP-43 in the cytoplasm of cells in a sample from a healthy subject (e.g., a subject that does not have a TDP-43 proteinopathy or a tauopathy, or a subject not at risk for a TDP-43 proteinopathy or a tauopathy). In some embodiments, a reference (e.g., a sample from a healthy subject) may be used to correlate and compare the amount and localization or TDP-43 obtained in the methods disclosed herein from a sample (e.g., a cerebrospinal sample). The reference may be obtained on the basis of one or more samples from normal or healthy subject, in particular samples which are not affected by a disease or disorder associated with the accumulation of TDP-43 in the nucleus and/or a tauopathy, either obtained from a subject or one or more different subjects. A reference may be determined empirically by testing a sufficiently large number of normal samples. A reference may also include immortalized cell lines. A sample may be sourced directly from a subject, or from a tissue library.

Provided herein are methods and compositions related to treating and/or preventing diseases or disorders associated TDP-43 proteinopathies and/or tauopathies in a subject by obtaining a sample (e.g., a sample with biological tissue, such as a cerebrospinal sample) comprising cells, identifying a genetic mutation in the DNA present in the sample, and administering to the subject (e.g., orally administering to the subject) a composition comprising a compound of Formula I or Formula II (e.g., nicotinamide riboside), and/or a compound of Formula III (e.g., pterostilbene). Also provided herein are methods of selecting a subject for therapy comprising administration of a composition comprising a compound of Formula I or Formula II (e.g., nicotinamide riboside), and/or a compound of Formula III (e.g., pterostilbene), the method comprising obtaining a sample comprising cells from the subject, and identifying a genetic mutation in the DNA present in the sample. If the cells within the sample comprise a genetic mutation, the method may further comprise selecting the subject for therapy comprising administration of a composition comprising a compound of Formula I or Formula II (e.g., nicotinamide riboside), and/or a compound of Formula III (e.g., pterostilbene).

In some embodiments, the genetic mutation is in the TARDBP gene, the gene that encodes TDP-43. The mutation in the TARDBP gene may be a loss of function mutation, truncation mutation, a deletion mutation, missense mutation, sense mutation, or any mutation that otherwise increases the amount of the TDP-43 in the cytoplasm. In some embodiments, the genetic mutation is in the N-terminus of the TDP-43 gene. In some embodiments, the genetic mutation is in the CHCHD10 gene. The mutation in the CHCHD10 gene may be a loss of function mutation, truncation mutation, a deletion mutation, missense mutation, nonsense mutation, or any mutation that otherwise increases the amount of the TDP-43 in the cytoplasm.

Identification of a mutation that increases the amount of TDP-43 in the cytoplasm may comprise any suitable method (e.g., sequencing method). The DNA in the sample may be sequenced by any known technique in the art, including, but not limited to, Maxam Gilbert sequencing, Sanger sequencing, shotgun sequencing, bridge PCR, or next generation sequencing methods, such as massively parallel signature sequencing (MPSS), polony sequencing, 454 pyrosequencing, Illumina (Solexa) sequencing, SOLiD sequencing, Ion torrent semiconductor sequencing, DNA nanoball sequencing, heliscope single molecule sequencing, single molecule real time (SMRT) sequencing, or nanopore DNA sequencing. The terms “Next Generation Sequencing” or “NGS” comprises all novel high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods. Multiple NGS platforms which are commercially available or which are mentioned in the literature can be used in the context of the methods disclosed herein, e.g. those described in detail in Zhang et al. 2011: The impact of next-generation sequencing on genomics. J. Genet Genomics 38 (3), 95-109; or in Voelkerding et al. 2009: Next generation sequencing: From basic research to diagnostics. Clinical chemistry 55, 641-658, each of which is hereby incorporated in its entirety.

Additionally, the methods disclosed herein may include identifying a pathological form of TDP-43 in the sample by obtaining a sample comprising cells from the subject and identifying a pathological form of TDP-43 in the sample, and if a pathological form of TDP-43 is present in the sample, administering to the subject (e.g., orally administering to the subject) a composition comprising a compound of Formula I or Formula II (e.g., nicotinamide riboside), and/or a compound of Formula III (e.g., pterostilbene). Also provided herein are methods of selecting a subject for therapy comprising administration of a composition comprising a compound of Formula I or Formula II (e.g., nicotinamide riboside), and/or a compound of Formula III (e.g., pterostilbene) by obtaining a sample comprising cells from the subject and identifying a pathological form of TDP-43 in the sample, and if a pathological form of TDP-43 is present in the sample, selecting the subject for therapy.

Specific biochemical modifications can give rise to pathological forms of TDP-43.

Therefore, identifying pathological forms of TDP-43, as disclosed herein, may comprise detecting post-translational modifications of TDP-43. For example, the identification of pathological forms of TDP-43 can comprise determining the phosphorylation state of TDP-43. In some embodiments, a pathological form of TDP-43 is identified if at least at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, or at least 70% of the TDP-43 in the cells of a sample are phosphorylated.

The methods may also comprise determining the ubiquitination state of TDP-43. In some embodiments, a pathological form of TDP-43 is identified if at least at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, or at least 70% of the TDP-43 in the cells of a sample are ubiquitinated. Exemplary methods for determining the phosphorylation or ubiquitination state of TDP-43 may be determined by any method known in the art, including but not limited to kinase activity assays, phospho-antibodies, western blot, or ELISA. The ubiquitination state of TDP-43 state may be elucidated by the use of ubiquitin proteasome system (UPS) reporters. UPS reporters usually comprise recognition sequences, are designed to specifically evaluate enzymatic activity of certain members of the UPS including the proteasome, E3 ubiquitin ligases, and deubiquitinating enzymes (DUBs).

The identification of a pathological form of TDP-43 can also comprise determining the presence or absence of C-terminal breakdown or cleavage fragments of TDP-43 in the cells in the sample. The C-terminal fragments can comprise fragments of from about 24 kD to about 26 kD. As provided below, the molecular signature of the TDP-43 protein can include the presence of C-terminal breakdown or cleavage products migrating at ^{˜}25 Kd, referred to as TDP-25. In some embodiments, the methods disclosed herein include obtaining a sample comprising cells from the subject, identifying a pathogenic form of TDP-43, such as TDP-25, and administering to the subject administering to the subject (e.g., orally administering to the subject) a composition comprising a compound of Formula I or Formula II (e.g., nicotinamide riboside), and/or a compound of Formula III (e.g., pterostilbene).

Actual dosage levels and administration regimen of the compositions disclosed herein may be varied so as to obtain an amount of a compound of Formula I or Formula II (e.g., nicotinamide riboside) and/or a compound of Formula III (e.g., pterostilbene) that is effective to achieve the desired therapeutic response for a particular patient, composition, and mode of administration, without being toxic to the patient.

In some embodiments, administration of the composition comprises administration of the composition in one or more dose(s). In some embodiments, administration of the composition comprises administration of the composition in one or more, five or more, ten or more, twenty or more, thirty or more, forty or more, fifty or more, one hundred or more, or one thousand or more dose(s). In some embodiments, the dose comprises at least 100 mg, at least 200 mg, at least 300 mg, at least 400 mg, at least 500 mg, at least 600 mg, at least 700 mg, at least 800 mg, at least 900 mg, at least 1000 mg, at least 1100 mg, at least 1200 mg, at least 1300 mg, at least 1400 mg, at least 1500 mg, at least 1600 mg, at least 1700 mg, at least 1800 mg, at least 1900 mg, at least 2000 mg, at least 2100 mg, at least 2200 mg, at least 2300 mg, at least 2400 mg, at least 2500 mg, at least 2600 mg, at least 2700 mg, at least 2800 mg, at least 2900 mg, or at least 3000 mg, of a compound of Formula I or Formula II (e.g., nicotinamide riboside). In some embodiments, the dose comprises at least 5 mg, at least 10, at least 20 mg, at least 30 mg, at least 40 mg, at least 50 mg, at least 60 mg, at least 80 mg, at least 100 mg, at least 120 mg, at least 140 mg, at least 160 mg, at least 180 mg, at least 200 mg, at least 220 mg, at least 240 mg, at least 260 mg, at least 280 mg, at least 300 mg, at least 320 mg, at least 340 mg, at least 360 mg, at least 380 mg, at least 400 mg, at least 500 mg, at least 600 mg, at least 700 mg, at least 800 mg, at least 900 mg, or at least 1000 mg of a compound of formula III (e.g., pterostilbene).

The compositions disclosed herein may be administered over any period of time effective to achieve the desired therapeutic response for a particular patient, composition, and mode of administration, without being toxic to the patient. The period of time may be at least 1 day, at least 10 days, at least 20 days, at least 30, days, at least 60 days, at least three months, at least six months, at least a year, at least three years, at least five years, or at least ten years. The dose may be administered when needed, sporadically, or at regular intervals. For example, the dose may be administered monthly, weekly, biweekly, triweekly, once a day, or twice a day.

The methods described herein may be performed or administered conjointly with any other treatment for TDP-43 proteinopathies or tauopathies. For example, approved therapeutic agents for Alzheimer's dementia, such as acetylcholinesterase inhibitors and memantine, have been used to treat other cognitive and behavioral symptoms in tauopathies. Therapeutic agents for the symptomatic treatment of Parkinson's disease (levodopa or dopamine agonists) are used for motor symptoms in tauopathies. For behavioral or psychopathological symptoms, treatment with antidepressants-especially selective serotonin reuptake inhibitors may be administered conjointly with the compositions disclosed herein. Additionally, the methods described herein may be performed or administered conjointly with any other treatment for ALS, including administration of drugs that delay the onset of ventilator-dependence or tracheostomy by lowering glutamate levels (e.g., Riluzole or Edaravone).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating or preventing a disease or disorder associated with TDP-43 cytoplasmic accumulation in a subject comprising cells exhibiting TDP-43 cytoplasmic accumulation, the method comprising administering to the subject a composition comprising nicotinamide riboside.

2. A method of treating or preventing an autophagy-associated disease or disorder in in a subject comprising cells exhibiting TDP-43 cytoplasmic accumulation, the method comprising administering to the subject a composition comprising nicotinamide riboside.

3. A method of treating or preventing an protein misfolding-associated disease or disorder in a subject comprising cells exhibiting TDP-43 cytoplasmic accumulation, the method comprising administering to the subject a composition comprising nicotinamide riboside.

4. A method of treating or preventing a neurodegenerative disease or disorder in a subject comprising cells exhibiting TDP-43 cytoplasmic accumulation, the method comprising administering to the subject a composition comprising nicotinamide riboside.

5. A method of treating or preventing a disease or disorder in a subject comprising cells exhibiting TDP-43 cytoplasmic accumulation, the method comprising administering to the subject a composition comprising nicotinamide riboside.

6. The method any one of claims 1 to 5, wherein the composition further comprises pterostilbene.

7. The method of any one of claims 1 to 5, further comprising administering to the subject a composition comprising pterostilbene.

8. The method of any one of claims 1 to 7, wherein the disease or disorder is a tauopathy.

9. The method of any one of claims 1 to 8, wherein the disease or disorder is Alzheimer's disease, amyotrophic lateral sclerosis, parkinsonism dementia complex of Guam, argyrophilic grain disease, dementia pugilistica, chronic traumatic encephalopathy, diffuse neurofibrillary tangles with calcification, Down's syndrome, familial British dementia, familial Danish dementia, frontal lobe dementia, frontotemporal lobular degeneration, Gertmann-Straussler-Scheinker disease, globular glial taupathies, white matter taupathy with globular glial inclusions, Guadeloupean parkinsonism with dementia, Guadeloupean progressive supranuclear palsy, multiple system atrophy, myotonic dystrophy, neurodegeneration with brain iron accumulation, Hallevorden-Spatz disease, pantothenate kinase-associated neurodegeneration, neurofibrillary tangle-predominant dementia, Niemann-Pick disease, type C, Picks's disease, prosencephalitic parkinsonism, prion protein cerebral amyloid angiopathy, progressive supranuclear palsy, SLC9 A6-related mental retardation, or subacute sclerosing panecephalitis.

10. The method of any one of claims 1 to 9, wherein the cells exhibiting TDP-43 accumulation are neurons.

11. The method of any one of claims 1 to 10, wherein at least 1% of the total cellular TDP-43 in the cells is in the cytoplasm of the cells.

12. The method of any one of claims 1 to 10, wherein at least 5% of the total cellular TDP-43 in the cells is in the cytoplasm of the cells.

13. The method of any one of claims 1 to 10, wherein at least 10% of the total cellular TDP-43 in the cells is in the cytoplasm of the cells.

14. The method of any one of claims 1 to 10, wherein at least 20% of the total cellular TDP-43 in the cells is in the cytoplasm of the cells.

15. The method of any one of claims 1 to 10, wherein at least 30% of total TDP-43 in the cells is in the cytoplasm of the cells.

16. The method of any one of claims 1 to 10, wherein at least 40% of total TDP-43 in the cells is in the cytoplasm of the cells.

17. The method of any one of claims 1 to 10, wherein at least 50% of total TDP-43 in the cells is in the cytoplasm of the cells.

18. The method of any one of claims 1 to 10, wherein the cells comprise a greater concentration of cytoplasmic TDP-43 than is present in the same cells in a healthy individual.

19. The method of any one of claims 1 to 18, wherein at least 1% of the neurons in the subject exhibit TDP-43 cytoplasmic accumulation.

20. The method of any one of claims 1 to 19, wherein the subject comprises a gene encoding a variant TDP-43 protein associated with increased cytoplasmic accumulation.

21. A method of treating or preventing a disease or disorder associated with TDP-43 cytoplasmic accumulation in a subject comprising a gene encoding a variant TDP-43 protein associated with increased cytoplasmic accumulation, the method comprising administering to the subject a composition comprising nicotinamide riboside.

22. A method of treating or preventing an autophagy-associated disease or disorder in in a subject comprising a gene encoding a variant TDP-43 protein associated with increased cytoplasmic accumulation, the method comprising administering to the subject a composition comprising nicotinamide riboside.

23. A method of treating or preventing a protein misfolding-associated disease or disorder in a subject comprising a gene encoding a variant TDP-43 protein associated with increased cytoplasmic accumulation, the method comprising administering to the subject a composition comprising nicotinamide riboside.

24. A method of treating or preventing a neurodegenerative disease or disorder in a subject comprising a gene encoding a variant TDP-43 protein associated with increased cytoplasmic accumulation, the method comprising administering to the subject a composition comprising nicotinamide riboside.

25. A method of treating or preventing a disease or disorder in a subject comprising cells exhibiting TDP-43 cytoplasmic accumulation, the method comprising administering to the subject a composition comprising nicotinamide riboside.

26. The method any one of claims 21 to 25, wherein the composition further comprises pterostilbene.

27. The method of any one of claims 21 to 25, further comprising administering to the subject a composition comprising pterostilbene.

28. The method of any one of claims 21 to 27, wherein the disease or disorder is a tauopathy.

29. The method of any one of claims 21 to 28, wherein the disease or disorder is Alzheimer's disease, amyotrophic lateral sclerosis, parkinsonism dementia complex of Guam, argyrophilic grain disease, dementia pugilistica, chronic traumatic encephalopathy, diffuse neurofibrillary tangles with calcification, Down's syndrome, familial British dementia, familial Danish dementia, frontal lobe dementia, frontotemporal lobular degeneration, Gertmann-Straussler-Scheinker disease, globular glial taupathies, white matter taupathy with globular glial inclusions, Guadeloupean parkinsonism with dementia, Guadeloupean progressive supranuclear palsy, multiple system atrophy, myotonic dystrophy, neurodegeneration with brain iron accumulation, Hallevorden-Spatz disease, pantothenate kinase-associated neurodegeneration, neurofibrillary tangle-predominant dementia, Niemann-Pick disease, type C, Picks's disease, prosencephalitic parkinsonism, prion protein cerebral amyloid angiopathy, progressive supranuclear palsy, SLC9 A6-related mental retardation, or subacute sclerosing panecephalitis.

30. The method of any one of claims 21 to 29, wherein the subject comprises cells exhibiting TDP-43 cytoplasmic accumulation.

31. A method of treating or preventing a disease or disorder associated with TDP-43 cytoplasmic accumulation in a subject, the method comprising:
(a) determining whether TDP-43 has accumulated in the cytoplasm of cells from a sample obtained from the subject; and
(c) if TDP-43 has accumulated in the cytoplasm of the cells, administering to the subject a composition comprising nicotinamide riboside.

32. A method of treating or preventing an autophagy-associated disease or disorder in a subject, the method comprising:
(a) determining whether TDP-43 has accumulated in the cytoplasm of cells from a sample obtained from the subject; and
(c) if TDP-43 has accumulated in the cytoplasm of the cells, administering to the subject a composition comprising nicotinamide riboside.

33. A method of treating or preventing an protein misfolding-associated disease or disorder in a subject, the method comprising:
(a) determining whether TDP-43 has accumulated in the cytoplasm of cells from a sample obtained from the subject; and
(c) if TDP-43 has accumulated in the cytoplasm of the cells, administering to the subject a composition comprising nicotinamide riboside.

34. A method of treating or preventing a neurodegenerative disease or disorder in a subject, the method comprising:
(a) determining whether TDP-43 has accumulated in the cytoplasm of cells from a sample obtained from the subject; and
(c) if TDP-43 has accumulated in the cytoplasm of the cells, administering to the subject a composition comprising nicotinamide riboside.

35. A method of treating or preventing a disease or disorder in a subject, the method comprising:
(a) determining whether TDP-43 has accumulated in the cytoplasm of cells from a sample obtained from the subject; and
(c) if TDP-43 has accumulated in the cytoplasm of the cells, administering to the subject a composition comprising nicotinamide riboside.

36. The method any one of claims 31 to 35, wherein the composition further comprises pterostilbene.

37. The method of any one of claims 31 to 35, further comprising administering to the subject a composition comprising pterostilbene if TDP-43 has accumulated in the cytoplasm of the subject.

38. The method of any one of claims 31 to 37, wherein the disease or disorder is a tauopathy.

39. The method of any one of claims 31 to 38, wherein the disease or disorder is Alzheimer's disease, amyotrophic lateral sclerosis, parkinsonism dementia complex of Guam, argyrophilic grain disease, dementia pugilistica, chronic traumatic encephalopathy, diffuse neurofibrillary tangles with calcification, Down's syndrome, familial British dementia, familial Danish dementia, frontal lobe dementia, frontotemporal lobular degeneration, Gertmann-Straussler-Scheinker disease, globular glial taupathies, white matter taupathy with globular glial inclusions, Guadeloupean parkinsonism with dementia, Guadeloupean progressive supranuclear palsy, multiple system atrophy, myotonic dystrophy, neurodegeneration with brain iron accumulation, Hallevorden-Spatz disease, pantothenate kinase-associated neurodegeneration, neurofibrillary tangle-predominant dementia, Niemann-Pick disease, type C, Picks's disease, prosencephalitic parkinsonism, prion protein cerebral amyloid angiopathy, progressive supranuclear palsy, SLC9 A6-related mental retardation, or subacute sclerosing panecephalitis.

40. The method of any one of claims 31 to 39, wherein the cells exhibiting TDP-43 accumulation are neurons.

41. The method of any one of claims 31 to 40, wherein at least 1% of the total cellular TDP-43 in the cells is in the cytoplasm of the cells.

42. The method of any one of claims 31 to 40, wherein at least 5% of the total cellular TDP-43 in the cells is in the cytoplasm of the cells.

43. The method of any one of claims 31 to 40, wherein at least 10% of the total cellular TDP-43 in the cells is in the cytoplasm of the cells.

44. The method of any one of claims 31 to 40, wherein at least 20% of the total cellular TDP-43 in the cells is in the cytoplasm of the cells.

45. The method of any one of claims 31 to 40, wherein at least 30% of total TDP-43 in the cells is in the cytoplasm of the cells.

46. The method of any one of claims 31 to 40, wherein at least 40% of total TDP-43 in the cells in the cytoplasm of the cells.

47. The method of any one of claims 31 to 40, wherein at least 50% of total TDP-43 in the cells is in the cytoplasm of the cells.

48. The method of any one of claims 31 to 47, wherein the step of determining the amount of TDP-43 in the cytoplasm comprises contacting the cells in the sample with an antibody specific for TDP-43.

49. The method of any one of claims 31 to 48, wherein the subject comprises a gene encoding a variant TDP-43 protein associated with increased cytoplasmic accumulation.

50. The method of any one of claims 31 to 49, wherein the sample comprising cells is a cerebrospinal sample.

51. A method of treating or preventing a disease or disorder associated with TDP-43 cytoplasmic accumulation in a subject, the method comprising
(a) determining whether the subject carries a pathological form of TDP-43, a variant TDP-43 protein associated with increased cytoplasmic accumulation or a gene encoding a variant TDP-43 protein associated with increased cytoplasmic accumulation; and
(c) if the subject carries a pathological form of TDP-43, a variant TDP-43 protein associated with increased cytoplasmic accumulation or a gene encoding a variant TDP-43 protein associated with increased cytoplasmic accumulation, administering to the subject a composition comprising nicotinamide riboside.
administering to the subject a composition comprising nicotinamide riboside.

52. A method of treating or preventing an autophagy-associated disease or disorder in in a subject, the method comprising
(a) determining whether the subject carries a pathological form of TDP-43, a variant TDP-43 protein associated with increased cytoplasmic accumulation or a gene encoding a variant TDP-43 protein associated with increased cytoplasmic accumulation; and
(c) if the subject carries a pathological form of TDP-43, a variant TDP-43 protein associated with increased cytoplasmic accumulation or a gene encoding a variant TDP-43 protein associated with increased cytoplasmic accumulation, administering to the subject a composition comprising nicotinamide riboside.

53. A method of treating or preventing an protein misfolding-associated disease or disorder in a subject, the method comprising
(a) determining whether the subject carries a pathological form of TDP-43, a variant TDP-43 protein associated with increased cytoplasmic accumulation or a gene encoding a variant TDP-43 protein associated with increased cytoplasmic accumulation; and
(c) if the subject carries a pathological form of TDP-43, a variant TDP-43 protein associated with increased cytoplasmic accumulation or a gene encoding a variant TDP-43 protein associated with increased cytoplasmic accumulation, administering to the subject a composition comprising nicotinamide riboside.

54. A method of treating or preventing a neurodegenerative disease or disorder in a subject, the method comprising
(a) determining whether the subject carries a pathological form of TDP-43, a variant TDP-43 protein associated with increased cytoplasmic accumulation or a gene encoding a variant TDP-43 protein associated with increased cytoplasmic accumulation; and
(c) if the subject carries a pathological form of TDP-43, a variant TDP-43 protein associated with increased cytoplasmic accumulation or a gene encoding a variant TDP-43 protein associated with increased cytoplasmic accumulation, administering to the subject a composition comprising nicotinamide riboside.

55. A method of treating or preventing a disease or disorder in a subject, the method comprising
(a) determining whether the subject carries a pathological form of TDP-43, a variant TDP-43 protein associated with increased cytoplasmic accumulation or a gene encoding a variant TDP-43 protein associated with increased cytoplasmic accumulation; and
(c) if the subject carries a pathological form of TDP-43, a variant TDP-43 protein associated with increased cytoplasmic accumulation or a gene encoding a variant TDP-43 protein associated with increased cytoplasmic accumulation, administering to the subject a composition comprising nicotinamide riboside.

56. The method any one of claims 51 to 55, wherein the composition further comprises pterostilbene.

57. The method of any one of claims 51 to 55, further comprising administering to the subject a composition comprising pterostilbene.

58. The method of any one of claims 51 to 57, wherein the disease or disorder is a tauopathy.

59. The method of any one of claims 51 to 58, wherein the disease or disorder is Alzheimer's disease, amyotrophic lateral sclerosis, parkinsonism dementia complex of Guam, argyrophilic grain disease, dementia pugilistica, chronic traumatic encephalopathy, diffuse neurofibrillary tangles with calcification, Down's syndrome, familial British dementia, familial Danish dementia, frontal lobe dementia, frontotemporal lobular degeneration, Gertmann-Straussler-Scheinker disease, globular glial taupathies, white matter taupathy with globular glial inclusions, Guadeloupean parkinsonism with dementia, Guadeloupean progressive supranuclear palsy, multiple system atrophy, myotonic dystrophy, neurodegeneration with brain iron accumulation, Hallevorden-Spatz disease, pantothenate kinase-associated neurodegeneration, neurofibrillary tangle-predominant dementia, Niemann-Pick disease, type C, Picks's disease, prosencephalitic parkinsonism, prion protein cerebral amyloid angiopathy, progressive supranuclear palsy, SLC9 A6-related mental retardation, or subacute sclerosing panecephalitis.

60. The method of any one of claims 51 to 59, wherein the subject comprises cells exhibiting TDP-43 cytoplasmic accumulation.

61. The method of any one of claims 51 to 60, wherein the gene is a TARDBP gene.

62. The method of claims 51 to 61, wherein the TDP-43 variant has a mutation at its N-terminus.

63. The method of claims 51 to 62, wherein the TDP-43 variant has a truncation mutation.

64. The method of any one of claims 51 to 63, wherein the pathological form is phosphorylated TDP-43.

65. The method of any one of claims 51 to 64, wherein identifying the pathological form of TDP-43 comprises contacting the cells in the sample with an antibody specific for the pathological form of TDP-43.

66. A method of selecting a subject for therapy comprising administration of a composition comprising nicotinamide riboside, the method comprising analyzing the amount of TDP-43 in the cytoplasm of cells in a sample from the subject and, if TDP-43 has accumulated in the cytoplasm of the cells, selecting the subject for therapy comprising administration of a composition comprising nicotinamide riboside.

67. The method of claim 66, wherein the composition further comprises pterostilbene.

68. The method of claim 66 or 67, wherein analyzing the cytoplasmic levels of TDP-43 comprise contacting sample with an antibody specific for TDP-43.

69. The method of any one of claims 1 to 68, wherein the administration of the composition comprises administering one or more doses of the composition.

70. The method of claim 69, wherein each dose of the composition comprises at least 800 mg of nicotinamide riboside.

71. The method of claim 69, wherein each dose of the composition comprises at least 1000 mg of nicotinamide riboside.

72. The method of claim 69, wherein each dose of the composition comprises at least 1200 mg of nicotinamide riboside.

73. The method of claim 69, wherein each dose of the composition comprises at least 1400 mg of nicotinamide riboside.

74. The method of claim 69, wherein each dose of the composition comprises at least 1600 mg of nicotinamide riboside.

75. The method of claim 69, wherein each dose of the composition comprises at least 1800 mg of nicotinamide riboside.

76. The method of claim 69, wherein each dose of the composition comprises at least 2000 mg of nicotinamide riboside.

77. The method of claim 69, wherein each dose of the composition comprises at least 2200 mg of nicotinamide riboside.

78. The method of any one of claims 69 to 77, wherein each dose of the composition comprises at least 60 mg of pterostilbene.

79. The method of any one of claims 69 to 77, wherein each dose of the composition comprises at least 100 mg of pterostilbene.

80. The method of any one of claims 69 to 77, wherein each dose of the composition comprises at least 200 mg of pterostilbene.

81. The method of any one of claims 69 to 77, wherein each dose of the composition comprises at least 300 mg of pterostilbene.

82. The method of any one of claims 69 to 77, wherein each dose of the composition comprises at least 400 mg of pterostilbene.

83. The method of any one of claims 69 to 77, wherein each dose of the composition comprises at least 500 mg of pterostilbene.

84. The method of any one of claims 69 to 77, wherein each dose of the composition comprises at least 600 mg of pterostilbene.

85. The method of any one of claims 69 to 84, wherein two or more doses of the composition are administered.

86. The method of any one of claims 69 to 84, wherein thirty or more doses of the composition are administered.

87. The method of any one of claims 69 to 84, wherein fifty or more doses of the composition are administered.

88. The method of any one of claims 69 to 84, wherein one hundred or more doses of the composition are administered.

89. The method of any one of claims 69 to 88, wherein the dose of the composition is administered at least once a week.

90. The method of any one of claims 69 to 88, wherein the dose is administered at least twice a week.

91. The method of any one of claims 69 to 88, wherein the dose is administered at least three times a week.

92. The method of any one of claims 69 to 88, wherein the dose is administered at least once a day.

93. The method of any one of claims 69 to 88, wherein the dose is administered at least twice a day.

94. The method of any one of claims 69 to 93, wherein the doses are administered for at least 7 days.

95. The method of any one of claims 69 to 93, wherein the doses are administered for at least 30 days.

96. The method of any one of claims 69 to 93, wherein the doses are administered for at least 60 days.

97. The method of any one of claims 69 to 93, wherein the doses are administered for at least 90 days.

98. The method of any one of claims 1 to 97, wherein the composition is formulated as a pill, a tablet, or a capsule.

99. The method of any one of claims 1 to 98, wherein the composition is administered orally.

100. The method of any one of claims 1 to 99, wherein the composition is self-administered.

101. The method of any one of claims 1 to 100, wherein the subject is human.

102. The method of any one of claims 1 to 101, further comprising the step of obtaining a sample from the subject.
